(19) United States
(12) Patent Application Publication
(10) Pub. No.: US 2010/0152688 A1
(43) Pub. Date: Jun. 17, 2010

(54) Wetness sensor insert

(76) Inventors: Julie Larsen Handwerker, Appleton, WI (US); Stephen Scott Harkness, Greenville, WI (US); Nathan Allen Genke, Wrightstown, WI (US); Thomas Michael Ales, III, Neenah, WI (US); Andrew Mark Long, Appleton, WI (US)

Correspondence Address: KIMBERLY-CLARK WORLDWIDE, INC.;Tara Pohlkotte, 2300 Winchester Rd., NEENAH, WI, 54956, US

(21) Appl. No.: 12/316,638

(22) Filed: Dec. 15, 2008

(51) Int. Cl. A61F 13/42

(52) U.S. Cl. 604/361

(57) ABSTRACT

An insert for sensing a bodily fluid or condition and for use in conjunction with an undergarment or a disposable absorbent article is provided, the insert including a first substrate having a first substrate article-facing surface and a first substrate body-facing surface, a second substrate having a second substrate body-facing surface, and an absorbent element disposed between the first and second substrates. The insert also includes two conductive elements disposed between the second substrate body-facing surface and the first substrate article-facing surface, wherein the conductive elements are adapted to be removably connectable to a signaling device. Also provided is an insert including a first substrate having a first substrate article-facing surface; and two conductive elements disposed on the first substrate, wherein each conductive element comprises conductive paper or tissue, and wherein the conductive elements are adapted to be removably connectable to a signaling device.

### BACKGROUND

Absorbent articles such as diapers, training pants, incontinence products, feminine hygiene products, swim undergarments, and the like conventionally include a liquid permeable body-side liner, a liquid impermeable outer cover, and an absorbent core. The absorbent core is typically located in between the outer cover and the liner for taking in and retaining liquids (e.g., urine) exuded by the wearer.

Many absorbent articles have been adapted for use in a training program, such as toilet training or enuresis control, or to provide indication of various medical, physical, or other conditions. Accordingly, various types of sensors and indicators, including moisture or wetness indicators, have been suggested for use in absorbent articles. Wetness indicators, for example, can include alarm devices that are designed to assist parents or attendants to identify a wet diaper condition quickly upon insult. The devices produce either a visual or an audible signal.

A large number of improvements to absorbent articles such as diapers, feminine care products, and incontinence products have been identified and developed, but not commercially exploited. Problems, however, have been encountered in producing and using such articles for training and/or notification purposes in that their uses in such functions represent a small percentage of sales when compared to the general market for disposable absorbent articles.

### SUMMARY

As caregivers undertake training, indication, and/or notification programs to address toilet training, enuresis control, incontinence monitoring, or condition monitoring, which apply across demographics, those caregivers would benefit by gaining greater access to products and guidance when needed, but being able to avoid the added costs of such items when they are not needed.

The problem with many of these improvements is cost. Incorporating some of these improvements into every absorbent article designed for daily use would be cost-prohibitive. For example, consumers can desire more training efficacy in an absorbent product. More training efficacy, however, can translate to a higher cost per product. A one cent cost increase per product is generally considered significant. One cent, however, does not purchase a large amount of training technology. Alternative improvements cannot be commercially exploited because they significantly alter the product form. Some improvements can make the product too thick; others can radically change the product's packaging requirements.

To solve these problems, a new sensing absorbent article system is required. The elements of the absorbent article system are generally a primary absorbent article with a secondary wetness sensing insert that are sold separately. Such a system allows a consumer to customize the primary absorbent article to meet the consumer's individual needs.

The present disclosure overcomes the problems by providing an insert for sensing a bodily fluid or condition and for use in conjunction with an undergarment or a disposable absorbent article, the insert including a first substrate having a first substrate article-facing surface and a first substrate body-facing surface, a second substrate having a second substrate body-facing surface, and an absorbent element disposed between the first and second substrates. The insert also includes two conductive elements disposed between the second substrate body-facing surface and the first substrate article-facing surface, wherein the conductive elements are adapted to be removably connectable to a signaling device.

The present disclosure also provides an insert for sensing a bodily fluid or condition and for use in conjunction with an undergarment or a disposable absorbent article, the insert including a first substrate having a first substrate article-facing surface; and two conductive elements disposed on the first substrate, wherein each conductive element comprises conductive paper or tissue, and wherein the conductive elements are adapted to be removably connectable to a signaling device.

The present disclosure also provides a disposable absorbent article capable of dual use by a wearer, the article including a liquid-impermeable outer cover having a front, a back and sides; an absorbent structure fixably juxtapositioned on the outer cover, the absorbent structure defining disposable containment and storage of body wastes; and a liquid-permeable bodyside liner covering the absorbent structure and backsheet. The article also includes a wetness sensing insert having a first substrate and a first end and a second end, the insert being disposed between the bodyside liner and the outer cover, wherein the insert includes two conductive elements adapted to be removably connectable to a signaling device, and wherein the insert is adapted to be removable from the article through an opening in the bodyside liner or the outer cover without removing the article from the wearer.

The present disclosure also provides a disposable absorbent article capable of dual use by a wearer, the article including a disposable absorbent article having a liquid-permeable bodyside liner and a wetness sensing insert placed on the bodyside liner and adapted to be removable from the article without removing the article from the wearer, wherein the insert includes a liquid-permeable first substrate and two conductive elements adapted to be removably connectable to a signaling device.

The present disclosure provides several attributes that are advantageous. An absorbent article system in which consumers can customize a basic absorbent article through the use of a supplementary insert enhances the efficiency of the absorbent-article-using process, helps to better care for the user, and can be more cost effective. Benefits of such a system to a business include a reduction of absorbent article machine capital expenditure, optimized manufacturing, more rapid evaluation and introduction of product improvements in the form of supplementary products, and potentially higher margins for supplementary products.

Other objects and advantages of the present disclosure will become more apparent to those skilled in the art in view of the following description and the accompanying drawings.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 representatively illustrates a side view of a pair of training pants with a mechanical fastening system of the pants shown fastened on one side of the training pants and unfastened on the other side of the training pants;

FIG. 2 representatively illustrates a plan view of the training pants of FIG. 1 in an unfastened, stretched and laid flat condition, and showing the surface of the training pants that faces away from the wearer, along with an optional arrangement of a wetness sensing insert;

FIG. 3 representatively illustrates a plan view similar to FIG. 2, but showing the surface of the training pants that faces the wearer when worn, and with portions cut away to show underlying features;

FIG. 4 representatively illustrates a schematic cross-section view of a particular aspect of the wetness sensing insert of the present disclosure;

FIG. 5 representatively illustrates a schematic cross-section view of another aspect of the wetness sensing insert of the present disclosure; and

FIG. 6 representatively illustrates a schematic exploded view of an aspect of the wetness sensing insert of the present disclosure.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

It is to be understood by one of ordinary skill in the art that the present discussion is a description of exemplary aspects of the present disclosure only, and is not intended as limiting the broader aspects of the present disclosure.

The present disclosure is generally directed to sensing inserts adapted to be attached to a signaling device that can be configured to indicate the presence of a body fluid at the insert or other changes in the condition of the product or wearer. The insert can be used in conjunction with a disposable absorbent article, undergarment, or other wearable article. The absorbent article can be, for instance, a diaper, a training pant, a pre-fastened pant, a swimming pant, an incontinence product, a feminine hygiene product, a medical garment, a bandage, or any other suitable article. Although generally referred to herein as a wetness sensing insert, the insert can be used to detect conditions and substances other than or in addition to wetness, as is described in more detail below. The wetness sensing insert label should not be taken as limiting the disclosure to wetness sensing.

One aspect of the disclosure described herein can be seen as a system of a primary absorbent article with a secondary wetness sensing insert. Such a system allows a consumer to customize the primary absorbent article to meet the consumer's individual needs. The absorbent article system can include any type of absorbent article at its core, including, but not limited to, a diaper, a training pant, a swim pant, a youth pant, a feminine hygiene product, an incontinence product, or the like. For purposes of illustration, and not for purposes of limitation, the absorbent article system is described as it would apply to a diaper. The same system, however, can be described using another suitable absorbent article, undergarment, garment, or other wearable article.

In the situation of childhood enuresis, enuretic alarms use wetness sensors to identify when a child has begun to wet and they wake them up to train them to wake up and use the restroom during the night. In the situation of adult enuresis, enuretic alarms use wetness sensors to identify when an adult has begun to wet to alert that adult or a caregiver of the wetness.

The sensors for wetness alarms, whether used in toilet training or enuretic situations, can be used on or in an absorbent article and held near the wearer's body, whether a diaper, pant, pad, or removable insert. When the child wets, the sensor will detect that wetness and sound waking the child up. Use of the insert in conjunction with an absorbent article of any kind should ease for the caregiver the difficulties associated with bed wetting and make the training process a more pleasant one. A removable insert or a pad can be more advantageous than a pant because there will not be any sizing issues like with current pant products.

The disclosure described herein can be used with any type of sensing article. In one type of sensing article used as a non-limiting example herein, wetness sensing absorbent articles can include an open circuit that becomes closed when a conductive fluid, such as a body fluid, is present in between a pair of conductive leads. Alternatively, wetness sensing absorbent articles can include a closed circuit that becomes open when a fluid, such as a body fluid, is present. Generally, the wetness sensing absorbent articles containing the circuit are disposable, meaning that they are designed to be discarded after a limited use rather than being laundered or otherwise restored for reuse.

The circuit contained within the wetness sensing absorbent articles of the present disclosure is configured to be attached to a signaling device. The signaling device can provide power to the circuit while also including some type of audible, visible, tactile, and/or electromagnetic signal that indicates to the user the presence of a body fluid. Although the wetness sensing insert 70 can itself be disposable, the signaling device can be reusable from article to article. In this regard, the present disclosure is also directed to different types of attachment modes that allow easy connection between the circuit in the wetness sensing insert 70 and the signaling device.

As described above, the circuit in combination with the signaling device can be configured to indicate the presence of a body fluid in contact with the wetness sensing insert 70. The particular targeted body fluid can vary depending upon the particular type of wetness sensing insert 70 and the desired application. For instance, in one aspect of the present disclosure, the wetness sensing insert 70 is associated with a diaper, a training pant, or the like, and the signaling device is configured to indicate the presence of urine. Alternatively, the signaling device can be configured to indicate the presence of a metabolite that would indicate the presence of a diaper rash. For adult incontinence products and feminine hygiene products, on the other hand, the signaling device can be configured to indicate the presence of yeast or of a particular constituent in urine or menses, such as a polysaccharide, a sugar, a protein, etc.

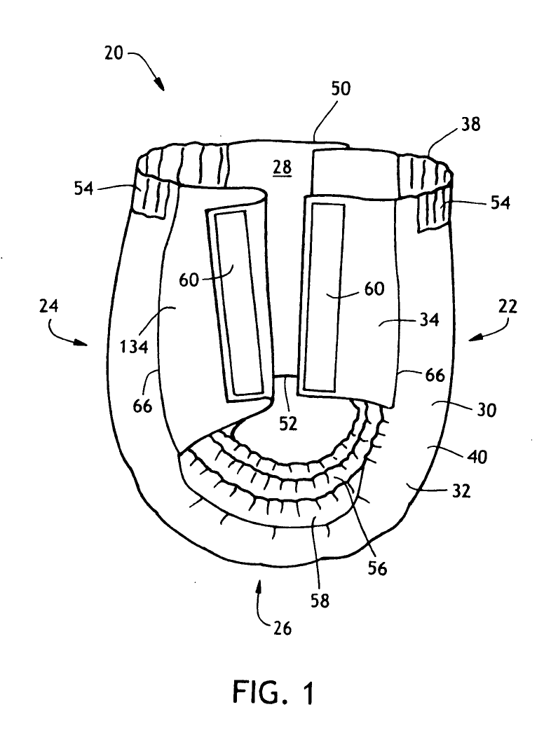

Referring now to the drawings and in particular to FIGS. 1-3, an absorbent article of the present disclosure is representatively illustrated in the form of children's toilet training pants and is indicated in its entirety by the reference numeral 20. The pants 20 include a wetness sensing insert 70 that is adapted to create a signal to the wearer upon urination, which can enhance a wearer's ability to recognize when urination is occurring. The pants 20 can be disposable, which refers to articles that are intended to be discarded after a limited period of use instead of being laundered or otherwise conditioned for reuse. It should also be understood that the present disclosure is suitable for use with various other absorbent articles intended for personal wear, including but not limited to diapers, feminine hygiene products, incontinence products, medical garments, surgical pads and bandages, other personal care or health care garments, and the like, without departing from the scope of the present disclosure. In addition, the present disclosure is suitable for use independent of an absorbent article, such as for use in conjunction with the wearer's own undergarment.

By way of illustration only, various materials and methods for constructing training pants such as the pants 20 of the various aspects of the present disclosure are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent that they are consistent (i.e., not in conflict) herewith. In addition, absorbent articles including a temperature change member are described in U.S. Pat. No. 5,681,298 to Brunner et al., the disclosure of which is incorporated herein by reference to the extent that it is consistent (i.e., not in conflict) herewith.

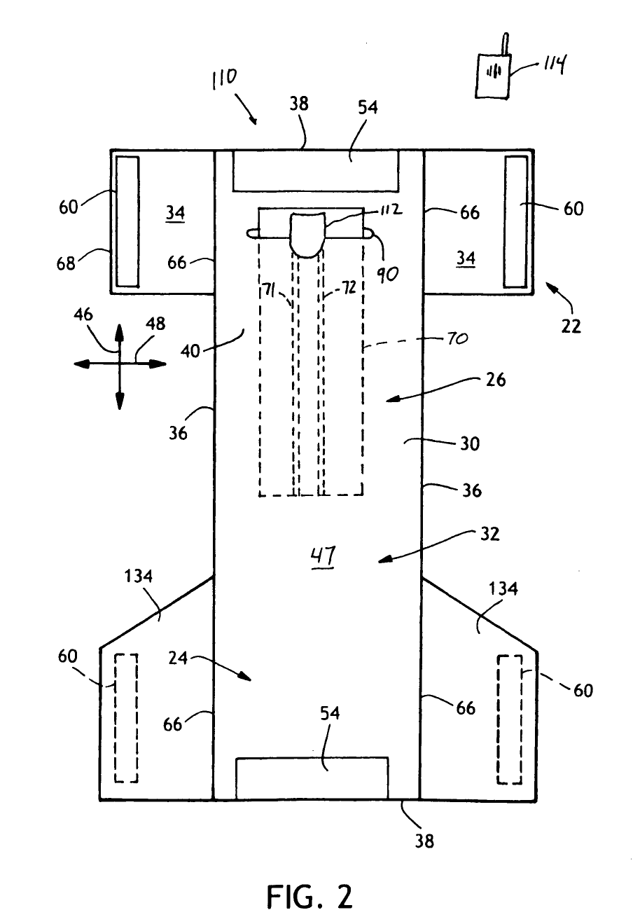

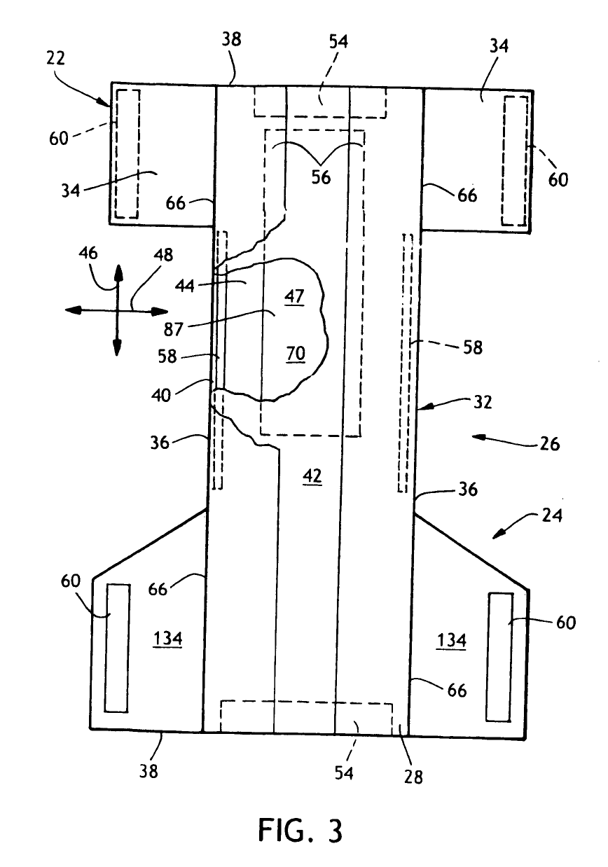

The pair of training pants 20 is illustrated in FIG. 1 in a partially fastened condition. The pants 20 define a longitudinal direction 46 and a lateral direction 48 perpendicular to the longitudinal direction as shown in FIGS. 2 and 3. The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The front and back waist regions 22, 24 includes those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The pants 20 also define an inner surface 28 adapted in use to be disposed toward the wearer, and an outer surface 30 opposite the inner surface 28. With additional reference to FIGS. 2 and 3, the pair of training pants 20 has a pair of laterally opposite side edges 36 and a pair of longitudinally opposite waist edges 38 (broadly, longitudinal ends).

The illustrated pants 20 can include an absorbent assembly, generally indicated at 32. For example, in the aspect of FIGS. 2 and 3, the pants 20 include a generally rectangular central absorbent assembly 32 and side panels 34, 134 formed separately from and secured to the central absorbent assembly. The side panels 34, 134 can be bonded along seams 66 to the absorbent assembly 32 in the respective front and back waist regions 22 and 24 of the pants 20. More particularly, the front side panels 34 can be permanently bonded to and extend laterally outward from the absorbent assembly 32 at the front waist region 22, and the back side panels 134 can be permanently bonded to and extend laterally from the absorbent assembly 32 at the back waist region 24. The side panels 34 and 134 can be bonded to the absorbent assembly 32 using attachment means known to those skilled in the art such as adhesive, thermal or ultrasonic bonding.

The front and back side panels 34 and 134, upon wearing of the pants 20, thus include the portions of the pants 20 that are positioned on the hips of the wearer. The front and back side panels 34 and 134 can be permanently bonded together to form the three-dimensional configuration of the pants 20, or be releasably connected with one another such as by a fastening system 60 of the illustrated aspects.

Suitable materials and structures are described further in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference in their entirety to the extent they are consistent (i.e., not in conflict) herewith. As is known in the art, the side panels 34, 134 can include elastic material or stretchable but inelastic materials.

The absorbent assembly 32 is illustrated in FIGS. 2 and 3 as having a rectangular shape. However, it is contemplated that the absorbent assembly 32 can have other shapes (e.g., hourglass, T-shaped, I-shaped, and the like) without departing from the scope of this disclosure. It is also understood that the side panels 34, 134 can alternatively be formed integrally with the absorbent assembly 32 without departing from the scope of this disclosure. In such a configuration, the side panels 34 and 134 and the absorbent assembly would include at least some common materials, such as the bodyside liner 42, outercover 40, other materials and/or combinations thereof.

The absorbent assembly 32 includes an outercover 40 and a bodyside liner 42 (FIGS. 2 and 3) in a superposed relation therewith. The liner 42 can be suitably joined to the outercover 40 along at least a portion of the longitudinal ends of the pants 20. The liner 42 can be suitably adapted, i.e., positioned relative to the other components of the pants 20, to contact the wearer's skin during wear of the pants. The absorbent assembly 32 also includes an absorbent body 44 (FIG. 3) disposed between the outercover 40 and the bodyside liner 42 for absorbing liquid body exudates. The liner 42 can be suitably joined to the outercover 40 along at least a portion of the longitudinal ends of the pants 20. The bodyside liner 42 and the outercover 40 can, for example, be attached to each other by adhesive, ultrasonic bonding, thermal bonding or by other suitable attachment techniques known in the art. Moreover, at least a portion of the absorbent body 44 can optionally be attached to the bodyside liner 42 and/or the outercover 40 utilizing the methods described above.

As mentioned above, the front and back side panels 34 and 134 can be releasably connected with one another such as by the fastening system 60 of the illustrated aspect. With the pants 20 in the fastened position as partially illustrated in FIG. 1, the front and back waist regions are connected together to define the three-dimensional pants configuration having a waist opening 50 and a pair of leg openings 52. The waist edges 38 (e.g., longitudinal ends) of the pants 20 are configured to encircle the waist of the wearer to define the waist opening 50 (FIG. 1) of the pants.

The fastening system 60 can include any refastenable fasteners suitable for absorbent articles, such as adhesive fasteners, cohesive fasteners, mechanical fasteners, or the like. In one aspect of the disclosure, the fastening system includes mechanical fastening elements for improved performance. Suitable mechanical fastening elements can be provided by interlocking geometric-shaped materials, such as hooks, loops, bulbs, mushrooms, arrowheads, balls on stems, male and female mating components, buckles, snaps, or the like.

The pants 20 can further include a pair of containment flaps 56 for inhibiting the lateral flow of body exudates. As illustrated in FIG. 1, the containment flaps 56 can be operatively attached to the pants 20 in any suitable manner as is well known in the art. In particular, suitable constructions and arrangements for the containment flaps 56 are generally well known to those skilled in the art.

To further enhance containment and/or absorption of body exudates, the pants 20 can include waist elastic members 54 in the front and/or back waist regions 22 and 24 of the pants 20. Likewise, the pants 20 can include leg elastic members 58, as are known to those skilled in the art. The waist elastic members 54 and the leg elastic members 58 can be formed of any suitable elastic material that is well known to those skilled in the art. For example, suitable elastic materials include sheets, strands or ribbons of natural rubber, synthetic rubber, or thermoplastic elastomeric polymers. In one aspect of the disclosure, the waist elastics and/or the leg elastics can include a plurality of dry-spun coalesced multi-filament spandex elastomeric threads.

The outercover 40 can suitably include a material that is substantially liquid impermeable. The outercover 40 can be provided by a single layer of liquid impermeable material, or more suitably include a multi-layered laminate structure in which at least one of the layers is liquid impermeable. In particular aspects, the outer layer can suitably provide a relatively cloth-like texture to the wearer. A suitable liquid impermeable film for use as a liquid impermeable inner layer, or a single layer liquid impermeable outercover 40 is a 0.025 millimeter (1.0 mil) polyethylene film. Alternatively, the outercover 40 can include a woven or non-woven fibrous web layer that has been totally or partially constructed or treated to impart the desired levels of liquid impermeability to selected regions that are adjacent or proximate the absorbent body.

The outercover 40 can also be stretchable, and in some aspects it can be elastomeric. For example, such an outercover material can include a 0.3 osy polypropylene spunbond that is necked 60 percent in the lateral direction 40 and creped 60 percent in the longitudinal direction 48, laminated with 3 grams per square meter (gsm) Bostik-Findley H2525A styrene-isoprene-styrene based adhesive to 8 gsm PEBAX 2533 film with 20 percent TiO₂ concentrate. The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent body 44. A suitable liquid permeable bodyside liner 42 is a nonwoven polyethylene/polypropylene bicomponent web having a basis weight of about 27 gsm; the web can be spunbonded or a bonded carded web. Optionally, the bodyside liner 42 can be treated with a surfactant to increase the wettability of the liner material.

Alternatively, the bodyside liner 42 can also be stretchable, and in some aspects it can be elastomeric. For instance, the liner 42 can be a non-woven, spunbond polypropylene fabric composed of about 2 to 3 denier fibers formed into a web having a basis weight of about 12 gsm that is necked approximately 60 percent. Strands of about 9 gsm KRATON G2760 elastomer material placed eight strands per inch (2.54 cm) can be adhered to the necked spunbond material to impart elasticity to the spunbond fabric. The fabric can be surface treated with an operative amount of surfactant, such as about 0.6 percent AHCOVEL Base N62 surfactant, available from ICI Americas, a business having offices in Wilmington, Del., U.S.A. Other suitable materials can be extensible biaxially stretchable materials, such as a neck stretched/creped spunbond. Reference is made to U.S. Pat. No. 6,552,245, issued Apr. 22, 2003, to Roessler et al., which is incorporated by reference herein to the extent that it is consistent (i.e., not in conflict) herewith.

An absorbent body 44 can be disposed on the outercover 40, for example, between the outercover 40 and the bodyside liner 42. The outercover 40 and the bodyside liner 42 can be joined together by any suitable means such as adhesives, ultrasonic bonds, thermal bonds, or the like. The absorbent body 44 can be in a variety of shapes and configurations as are known in the art, such as rectangular, hourglass shaped, I-shaped, and the like. Further, at least a portion of the absorbent body 44 can optionally be attached to the bodyside liner 42 and/or the outercover 40 utilizing the methods described above.

The absorbent body 44 is suitably compressible, conformable and capable of absorbing and retaining liquid body exudates released by the wearer. For example, the absorbent assembly can include a matrix of absorbent fibers, and more suitably cellulosic fluff, such as wood pulp fluff, and superabsorbent particles. As an alternative to wood pulp fluff, synthetic fibers, polymeric fibers, meltblown fibers, short cut homofil bicomponent synthetic fibers, or other natural fibers can be used. Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid.

The absorbent body 44 can have a density within the range of about 0.10 to about 0.5 grams per cubic centimeter and can be wrapped or encompassed by a suitable tissue or nonwoven wrap for maintaining the integrity and/or the shape of the absorbent assembly.

In one aspect, the absorbent body 44 can be stretchable so as not to inhibit the stretchability of other components to which the absorbent body can be adhered, such as the outercover 40 and/or the bodyside liner 42.

In some aspects, a surge management layer (not shown) can be included in the pants 20. The surge management layer can be positioned in the pants 20 in a variety of locations as is known in the art. For example, the surge management layer can be proximate the absorbent body 44, for example between the absorbent body 44 and the bodyside liner 42, and attached to one or more components of the pants 20 by methods known in the art, such as by adhesive, ultrasonic or thermal bonding. In addition, the surge management layer can be positioned in the pants 20 relative to the wetness sensing insert 70 in a variety of ways. For instance, the surge management layer can be disposed toward the liner 42 relative to the wetness sensing insert 70, or the surge management layer can be disposed toward the absorbent body 44 relative to the wetness sensing insert 70.

A surge management layer helps to decelerate and diffuse surges or gushes of liquid that can be rapidly introduced into the absorbent body 44. Desirably, the surge management layer can rapidly accept and temporarily hold the liquid prior to releasing the liquid into the storage or retention portions of the absorbent body 44.

The wetness sensing insert 70 described above and illustrated in FIGS. 2-6 can be used in conjunction with and disposable absorbent article or other garment, undergarment, or wearable article. The use of a wetness sensing insert 70 can result in reduced delay in signaling to a wearer or caregiver. Construction of the wetness sensing insert 70 allows for placement of conductive materials near the body of the wearer to allow for quick sensing and indication, which can be critical in certain intended uses.

One such wetness sensing insert 70 is the wetness sensing insert 70 described herein. In this regard, as shown in FIGS. 2-6, the wetness sensing insert 70 includes a first conductive element 71 spaced from a second conductive element 72. In this aspect of the present disclosure, the conductive elements 71 and 72 extend from one end of the wetness sensing insert 70 to the other end without intersecting. The conductive elements 71 and 72 can comprise any suitable conductive material, such as a conductive thread, a conductive foil, vapor or laminate foils, printed ink including metal and/or carbon on a carrier paper or film, or a conductive paper or tissue such as that described in co-pending U.S. patent applications Ser. Nos. 11/888,258 and 11/888,334. The first conductive element 71 cannot intersect the second conductive element 72 to form an open circuit that can be closed, for instance, when a conductive fluid is positioned in between the conductive elements.

The form of each conductive element 71 and 72 can be tailored to the intended use of the wetness sensing insert 70 such that the conductive elements 71 and 72 are optimized to detect wetness, pH, or temperature, for example, or to analyze an analyte. In such aspects of the present disclosure, the first conductive element 71 and the second conductive element 72 can be connected to a sensor within the wetness sensing insert 70. The sensor can be used to sense changes in temperature or can be used to sense the presence of a particular substance, such as a metabolite.

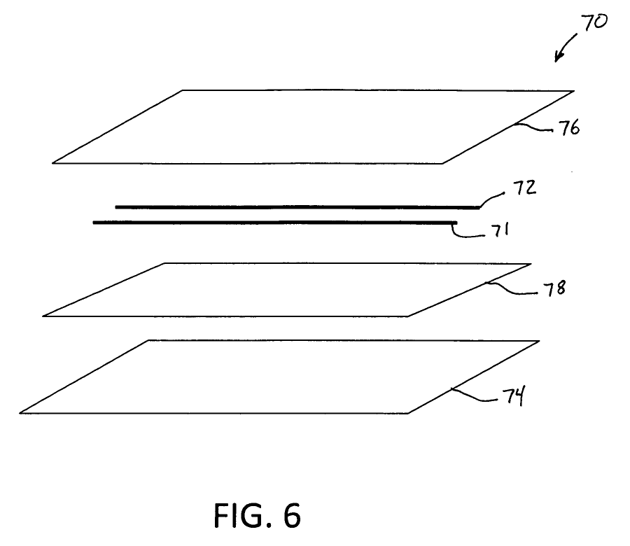

In the aspect of the present disclosure shown in FIGS. 2 and 6, the conductive elements 71 and 72 extend the entire length of the wetness sensing insert 70. It should be understood, however, that in other aspects of the present disclosure the conductive elements 71 and 72 can extend only partially along the wetness sensing insert 70 or can extend to any particular place on the wetness sensing insert 70 where a body fluid is intended to be sensed.

As best illustrated in FIG. 6, the wetness sensing insert 70 includes a first substrate 74 having a body-facing side and an article-facing side. The conductive elements 71 and 72 can be incorporated into or onto the first substrate 74 at any suitable location as long as the conductive elements 71 and 72 are positioned so as to contact a body fluid that is contacted by the wetness sensing insert 70. In this regard, the conductive elements 71 and 72 generally lie on or above the body-facing side of the first substrate 74. The wetness sensing insert 70 can also include a second substrate 76 having a body-facing side and an article-facing side. In this aspect, the conductive elements 71 and 72 are generally disposed between the first and second substrates 74 and 76. The conductive elements 71 and 72 can be attached to the first and/or second substrates 74 and 76, and the first and second substrates 74 and 76 to each other, using adhesive or by any other suitable means.

Because of its positioning between the body and the conductive elements 71 and 72, the second substrate 76 generally comprises a liquid-permeable nonwoven to allow bodily fluid to pass through the second substrate to contact the conductive elements 71 and 72. The first substrate 74 can be either liquid-permeable, liquid-impermeable, or somewhere in between depending on how much bodily fluid is intended to pass therethrough. For example, for a wetness sensing insert 70 designed to be used in conjunction with a disposable absorbent article, the first substrate 74 would typically be liquid-permeable. In another example, for a wetness sensing insert 70 designed to be used in conjunction with a regular undergarment, the first substrate 74 would typically be liquid-impermeable to prevent or at least slow bodily fluid from contacting the undergarment.

In an alternate aspect of the present disclosure, the wetness sensing insert 70 can include an absorbent element 78 disposed between the first and second substrates 74 and 76. In one aspect, the absorbent element 78 comprises the same materials described above with respect to the absorbent body 44. In these aspects, the conductive elements 71 and 72 can be positioned on any surface of the first substrate 74, second substrate 76, or absorbent element 78 as long as the conductive elements 71 and 72 can contact bodily fluid.

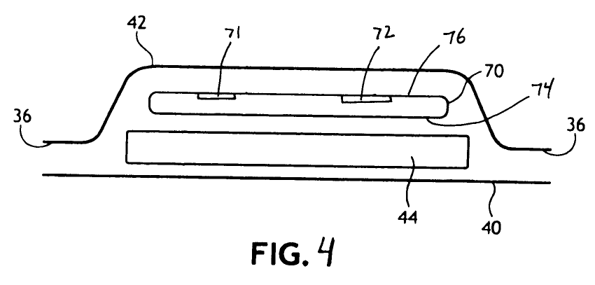

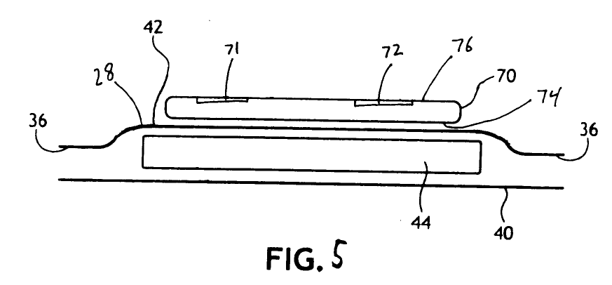

As illustrated in FIGS. 4 and 5, when used in conjunction with training pants 20, the wetness sensing insert 70 can be positioned between the bodyside liner 42 and the absorbent body 44 (FIG. 4), or on the inner surface 28 of the bodyside liner 42 (FIG. 5). In either position, the wetness sensing insert 70 can be held in place by friction, gravity, bonding, sewing, or by any suitable adhesive. The wetness sensing insert 70 can be positioned by a manufacturer or by a consumer/wearer, as described herein.

The conductive elements 71 and 72 can be connected directly to a signaling device 110, either through direct or indirect contact. The conductive elements 71 and 72 can be attached to conductive pad members (not shown), which can be of the type described in U.S. Pat. No. 7,394,391. In this aspect, the pad members are provided for making a reliable connection between the open circuit formed by the conductive elements to a signaling device that is intended to be installed on the chassis by the consumer or manufacturer. The pad members create a zone for connecting the signaling device and the conductive leads or elements.

The position of the conductive pad members on the wetness sensing insert 70 can vary depending upon where it is desired to mount the signaling device 110. For instance, the conductive pad members can be positioned on one end of the wetness sensing insert 70. In another aspect, the wetness sensing insert 70 can include conductive pad members positioned at each end of each conductive element 71 and 72. In still other aspects of the present disclosure, it should be understood that the pad members can be located along a side of the wetness sensing insert 70.

The position of the conductive pad members within the multiple layers of the wetness sensing insert 70 can also vary depending upon where it is desired to connect the signaling device 1 10 and the type of attachment mode used to make a connection with the signaling device 110. As described above, the pad members are electrically connected to the conductive elements 71 and 72. Thus, in one aspect, the pad members are positioned toward the body-facing side of the first substrate 74. Positioning the pad members below at least one layer of material can provide various advantages in some aspects of the present disclosure. For instance, locating the pad members below at least one layer of material within the wetness sensing insert 70 protects the pad members during shipping and storage and from forming a short circuit during use especially if the pad members are located adjacent one another. Another benefit to placing the pad members under at least one layer of material is the ability to easily manufacture the wetness sensing insert 70 at high machine speeds.

It should be understood, however, that in other aspects of the present disclosure the conductive pad members can be positioned at an article-facing surface of the wetness sensing insert 70.

Connection between the conductive materials and an active member such as a sensor, a signaling device 110, other electronics, a power supply, an indicator, etc., can be very flexible in design. Alterations can be made to the sandwich materials to allow for easier connection. Potential alterations include perforations, die cuts, and phased basis weights. Conductive materials can be exposed through one of the substrates 74 and 76 for ease of connection. The substrates 74 and 76 and the conductive material can be designed or placed differently to enhance connection. For example, the conductive materials can be longer, situated differently in the wetness sensing insert 70, or tapered, regardless of the article with which the wetness sensing insert 70 will be used.

The connection design described herein also allows for flexibility in the design of an active member to be used in conjunction with the wetness sensing insert 70.

Having more connection flexibility will increase the design and ergonomic characteristics of the wetness sensing insert 70 and the active member. The active member can be flexible with respect to input and output based on the intended use of the wetness sensing insert 70 and can vary depending on age of user, gender, etc. and can include a wetness sensor, indicator, tracker, and/or predictor, or a condition monitor, indicator, tracker, and/or predictor.

Referring to FIG. 2 for exemplary purposes, a signaling device 110 (as depicted by ref. numerals 112 and 114) is shown attached to the wetness sensing insert 70.

As shown, in this aspect of the present disclosure, the signaling device can include a transmitter 112 and a receiver 114. The transmitter 112 includes a pair of opposing terminals that are electrically connected to the corresponding conductive elements. When a body fluid is present at the wetness sensing insert 70, the open circuit formed by the conductive elements 71 and 72 is closed which, in turn, activates the signaling device 110. In particular, in this aspect of the present disclosure, the transmitter 112 sends a wireless signal to the receiver 114 which then indicates to a user that a body fluid is present at the wetness sensing insert 70. In other various aspects of the present disclosure, any chemical or physical reaction that can change conductivity can also be sensed by this type of system.

Examples include conductivity changes associated with electrochemical sensors such as those described in co-pending and co-assigned U.S. patent application Ser. No. 11/314,438, filed on Dec. 21, 2005, by Ales et al. and entitled “Personal Care Products with Microchemical Sensors for Odor Detection,” which is incorporated herein by reference to the extent it is consistent (i.e., not in conflict) herewith.

The signaling device 110 can emit an audible signal or a visual signal to indicate to the user that the circuit has been closed. The audible signal, for instance, can be as simple as one or more beeps to perhaps emitting a musical tune. Similarly, if the signaling device 110 issues a visible signal, the visible signal can comprise one light, a few lights, or an interactive display. In still another aspect of the present disclosure, the receiver 114 of the signaling device 110 can be configured to vibrate when the circuit within the wetness sensing insert 70 is closed.

In the aspect of the present disclosure shown in FIG. 2, the signaling device 110 includes a transmitter 112 in combination with a receiver 114. It should also be understood, however, that the signaling device 110 can comprise a single unit that remains attached to the wetness sensing insert 70. For example, the signaling device 110 can be mounted on the wetness sensing insert 70 and issue a visible signal and/or an audible signal from the insert itself.

The wetness sensing insert 70 can include fastening elements to allow the wetness sensing insert 70 to be affixed to a disposable absorbent article or to an undergarment, garment, or other wearable article. Suitable fastening elements include adhesives, cohesives, and hook-and-loop. In the case of a regular undergarment or garment, the wetness sensing insert 70 is desirably removably attached to the garment. In the case of a disposable absorbent article, the wetness sensing insert 70 can be either removably or non-removably attached to the article. In the latter case, the wetness sensing insert 70 can be liquid-permeable and/or breathable to allow for unaltered product performance. The wetness sensing insert 70 can be manufactured in a variety of sizes to match various garments and articles, or the wetness sensing insert 70 can be manufactured in a one-size arrangement that is of an appropriate size to effectively cover the article or garment in a large percentage of our article/garment forms. The width of the wetness sensing insert 70 should be enough to allow the wetness sensing insert 70 to be positioned without extending into the leg openings of the article/garment. The length of the wetness sensing insert 70 should be long enough to reach insult target zones for either gender.

For the aspect of the wetness sensing insert 70 without an absorbent element 78, the manufacturing process can be easier because there will be no process disruption due to absorbent materials. In addition, the wetness sensing insert 70 can be manufactured in a large or wide format with many conductive networks laid in at one time, and then further converted to smaller wetness sensing insert 70 form by slitting, spooling, and/or die cutting to produce individual wetness sensing inserts 70.

For the aspect of the wetness sensing insert 70 with an absorbent element 78, the wetness sensing insert 70 can have applicability over several disposable absorbent and garment product forms. Certain sizes of wetness sensing inserts 70 can be designed for insertion and use in garment products, while others can be manufactured specifically for use in pants and diapers. In some applications, a reduced absorbent capacity can be desired. Wetness detection could mean reduced superabsorbent material or fluff for single void use or wetness detection and quick indication, such as for use in conjunction with incontinence.

In other applications, the article or garment can remain on the wearer while the wetness sensing insert 70 is removed and replaced. In the case of a disposable absorbent article, the article design can be altered to allow for the best placement of a pocket 90 in which to insert the wetness sensing insert 70. In an alternate aspect, the wetness sensing insert 70 can be disposed between the bodyside liner 42 and the outer cover 40, and designed such that the wetness sensing insert 70 is removable from the article through an opening 90 in the bodyside liner 42 or in the outer cover 40 while the article is being worn, without removing the article from the wearer. A specific example is illustrated in FIG. 2 with pocket or opening 90 in the outer cover 40 through which the wetness sensing insert extends. In alternate aspects, the opening can be in either the bodyside liner 42 or the outer cover 40, and in the front waist region 22 or the back waist region 24, or in any other suitable location.

The wetness sensing insert 70 described herein allows a manufacturer to produce an article or a garment and a wetness sensing insert 70 and package these separately. Separate packaging allows for flexibility in retail department placement such as in pharmacy, baby care, adult care, etc. Each separate package can include a description of the condition intended to be addressed by the product in that package, a description of the primary function of that product, a description of the entire system, or some combination thereof. The descriptions can be placed on the packaging, in the packaging, or some combination of both. The descriptions can allow a consumer to become educated as to the conditions that typically dictate the use of an absorbent article, and the further conditions that can be associated with or experienced in conjunction with the use of an absorbent article. Such education allows a consumer to make informed decisions as to which wetness sensing insert 70 to purchase such that the consumer can assemble a customized article or garment to address various conditions.

Consumer education can be further enhanced by providing indicia on the packaging to indicate various different but associated components of the system. The indicia can include graphic design features, text, branding, or any other suitable indicia. For example, all of the packages can exhibit similar graphic design components and branding to identify their association, yet have different coloring and illustrations to identify their differences. These indicia can help a consumer to quickly discern which products are needed for a particular purpose.

In other words, the system described herein allows a consumer to customize an absorbent article or a garment on an as-needed basis to address whatever conditions the wearer is experiencing. Consumers desire a high quality, consistent product at a good price with the ability to upgrade for certain circumstances.

For a manufacturer, incorporating improvements into, for example, the wetness sensing insert 70 rather than in a basic absorbent article, allows the manufacturer to make minimal changes to absorbent article machines, resulting in lower capital expenditures and optimizing manufacturing. In addition, researchers can focus on product improvements without considering the effect of the improvements on absorbent article machines. The manufacturer can contract manufacturing of secondary products and enter into joint marketing agreements with makers of related items. Wetness sensing inserts 70 can be rapidly introduced and evaluated, and need not be as cost sensitive as improvements incorporated into every absorbent article. Finally, customized wetness sensing inserts 70 allow the manufacturer to more effectively meet global needs.

Other objects and advantages of the present disclosure will become more apparent to those skilled in the art in view of the following description and the accompanying drawings.

The disclosure has been described with reference to various specific and illustrative embodiments and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

We claim:

1. An insert for sensing a bodily fluid or condition and for use in conjunction with an undergarment or a disposable absorbent article, the insert comprising:
a first substrate having a first substrate article-facing surface and a first substrate body-facing surface;
a second substrate having a second substrate body-facing surface;
an absorbent element disposed between the first and second substrates;
two conductive elements disposed between the second substrate body-facing surface and the first substrate article-facing surface, wherein the conductive elements are adapted to be removably connectable to a signaling device.

2. The insert of claim 1, wherein each conductive element comprises conductive paper or tissue.

3. The insert of claim 1, wherein the second substrate is liquid-permeable.

4. The insert of claim 1, wherein the first substrate is liquid-impermeable.

5. The insert of claim 1, wherein the insert is adapted to be disposed in a pocket in a disposable absorbent article.

6. The insert of claim 1, wherein the insert is adapted to be fastened to a disposable absorbent article.

7. The insert of claim 7, wherein the insert is adapted to be fastened to the disposable absorbent article using adhesive.

8. An insert for sensing a bodily fluid or condition and for use in conjunction with an undergarment or a disposable absorbent article, the insert comprising:
a first substrate having a first substrate article-facing surface; and
two conductive elements disposed on the first substrate, wherein each conductive element comprises conductive paper or tissue, and wherein the conductive elements are adapted to be removably connectable to a signaling device.

9. The insert of claim 8, further comprising a second substrate having a second substrate body-facing surface and an absorbent element disposed between the first and second substrates.

10. The insert of claim 8, wherein the two conductive elements are disposed between the second substrate body-facing surface and the first substrate article-facing surface.

11. The insert of claim 8, wherein the insert is adapted to be disposed in a pocket in a disposable absorbent article.

12. The insert of claim 8, wherein the insert is adapted to be fastened to a disposable absorbent article.

13. The insert of claim 12, wherein the insert is adapted to be fastened to the disposable absorbent article using adhesive.

14. A disposable absorbent article capable of dual use by a wearer, the article comprising:
a liquid-impermeable outer cover having a front, a back and sides;
an absorbent structure fixably juxtapositioned on the outer cover, the absorbent structure defining disposable containment and storage of body wastes;
a liquid-permeable bodyside liner covering the absorbent structure and backsheet; and
a wetness sensing insert having a first substrate and a first end and a second end, the insert being disposed between the bodyside liner and the outer cover, wherein the insert includes two conductive elements adapted to be removably connectable to a signaling device, and wherein the insert is adapted to be removable from the article through an opening in the bodyside liner or the outer cover without removing the article from the wearer.

15. The article of claim 14, wherein each conductive element comprises conductive paper or tissue.

16. The article of claim 14, wherein the wetness sensing insert includes an absorbent element.

17. A disposable absorbent article capable of dual use by a wearer, the article comprising:
a disposable absorbent article having a liquid-permeable bodyside liner; and
a wetness sensing insert placed on the bodyside liner and adapted to be removable from the article without removing the article from the wearer, wherein the insert comprises:
a liquid-permeable first substrate, and
two conductive elements adapted to be removably connectable to a signaling device.

18. The article of claim 17, wherein each conductive element comprises conductive paper or tissue.

19. The article of claim 17, wherein the wetness sensing insert includes an absorbent element.
